(19) United States
(12) Patent Application Publication
(10) Pub. No.: US 2020/0022945 A1
(43) Pub. Date: Jan. 23, 2020

(54) RAPIDLY DISSOLVING PHARMACEUTICAL COMPOSITIONS AND METHOD OF MANUFACTURING

(71) Applicant: CONTANGO LABS INC. D/B/A ANEWSHA, Hermosa Beach, CA (US)

(72) Inventor: Chris Thomas SWARTOUT, East Marion, NY (US)

(21) Appl. No.: 16/516,189

(22) Filed: Jul. 18, 2019

Related U.S. Application Data
(60) Provisional application No. 62/764,130, filed on Jul. 18, 2018.

(51) Int. Cl. A61K 31/352; A61K 36/185; A61K 9/00; A61K 31/05; A61K 9/20

(52) U.S. Cl. CPC A61K 31/352; A61K 36/185; A61K 9/2018; A61K 31/05; A61K 9/0056

(57) ABSTRACT

The present disclosure provides rapidly dissolving oral compositions comprising an effective amount of a cannabinoid and a pharmaceutically acceptable excipient or mixtures of excipients and methods of preparing such rapidly dissolving oral compositions.

### CROSS-REFERENCE TO RELATED APPLICATIONS

This application claims priority to U.S. Provisional Patent Application No. 62/764,130, filed Jul. 18, 2018, the disclosure of which is hereby incorporated by cross-reference in its entirety.

### BACKGROUND OF DISCLOSURE

### Field

The present disclosure provides rapidly dissolving oral compositions comprising cannabinoids and a pharmaceutically acceptable excipient or mixtures of excipients and a method of preparing the rapidly dissolving oral composition, wherein the compositions are designed to dissolve in the oral cavity of the patient.

### Technical Background

Cannabis is a highly variable genus of flowering plant that contains hundreds of different biologically active chemicals, including cannabinoids and terpenes. Cannabidiol (CBD), Δ9-tetrahydrocannabinol (THC), other phytocannabinoids and phytochemicals produced by Cannabis plants act on cannabinoid receptors located throughout the body of humans, mammals, birds, fish, and reptiles.

Both THC and CBD have been recognized has having medical benefits. THC can help improve pain, increase appetite, improve nausea and vomiting associated with cancer chemotherapy, and can have effects on mood, memory, cognition, and perception, among other benefits. CBD, which is a non-psychoactive cannabinoid, can help improve, for example, inflammation, anxiety, and depression.

Cannabis can be consumed in a number of ways, including by inhalation and ingestion. However, the rate of absorption and onset time of the effects of Cannabis are dependent on the consumption method. Inhaling Cannabis can have an almost immediate effect, but the medical effects can last for a short amount of time. However, for patients who prefer or need to consume Cannabis by ingestion, it can take hours to experience the medical effects of Cannabis. Furthermore, some patients who have difficulty swallowing or need to take medication without water can benefit from formulations that can rapidly dissolve in the mouth and also have a good taste or no taste.

Thus, there is a need for new and improved formulations for consuming Cannabis orally. Described herein are rapidly dissolving solid dosage compositions containing cannabinoids as active ingredients for targeted fast acting therapeutic use. The compositions of the disclosure can rapidly dissolve when administered in the oral cavity, thus increasing the bioavailability of cannabinoids in the receptor systems. The compositions also have favorable organoleptic properties, making them tolerable to consume by dissolving in the mouth.

### BRIEF SUMMARY OF THE DISCLOSURE

The present disclosure relates to rapidly dissolving oral compositions comprising cannabinoids and a pharmaceutically acceptable excipient or mixture of excipients and methods of making the compositions.

One aspect of the present disclosure provides a rapidly dissolving oral composition comprising an effective amount of a cannabinoid and a pharmaceutically acceptable excipient or mixtures of excipients.

In some embodiments of the disclosure, the cannabinoid is tetrahydrocannabinol (THC), delta-9-tetrahydrocannabivarin (THCv), 10-Ehtoxy-9-hydroxy-delta-6a-tetrahydrocannabinol, 8,9-dihydroxy-delta-6a-tetrahydrocannabinol, delta-8-tetrahydrocannabinol, delta-8-tetrahydrocannabinolic acid, delta-9-tetrahydrocannabinol-C4 (THC-C4), delta-9-tetrahydrocannabinoic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorolic acid (THCA-C1), delta-9-tetrahydrocannabivarinic acid (THCVA), 10-Oxo-delta-6a-tetrahydrocannabinol (OTHC), dehydrocannabifuran (DCBF), delta-9-cis-tetrahydrocannabinol (cis-THC), trhydroxy-delta-9-tetrahydrocannabinol (triOH-THC), 3,4,5,6,-tetrahydro-7-hydroxy-alpha-alpha-2-trimethyl-9-n-propyl-2, cannabidiol (CBD), cannabinol (CBN), cannabichromene (CBC), cannabichromenic acid (CBCA), cannabicyclol (CBL), cannabicyclolic acid (CBLA), cannabicyclovarin (CBLV), cannabivarin (CBV), cannabidivarin (CBDVa), cannabinodiol (CBND), cannabielsion (CBE), cannabicyclol (CBL), cannabicyloic acid (CBLA), cannabitriol (CBT), cannabidiol monomehylerther (CBDM), cannabidiolic acid (CBDA), cannabidiorcol (CBD-C1), cannabielsoic acid B (CBEA-B), cannabielsin (CBE), cannabielsoin acid A (CBEA-A), cannabigerol (CBG), cannabigerol monomethlether (CBGM), cannabigerolic acid (CBGA), cannabigerolic acid (CBGA), cannabigerolic acid monomethlether (CBGAM), cannabigerovarin (CBGV), cannabigerovarinic acid (CBGVA), cannabinodiol (CBND), cannabinodivarin (CBVD), cannabinol methlether (CBNM), cannabinol-C2 (CBN-C2), cannabinol-C4 (CBN-C4), cannabinolic acid (CBNA), cannabiorcool (CBN-C1), cannabivarin (CBV), cannaitriol (CBT), cannabitriolvarin (CBTV), cannabichromanon (CBCF), cannabifuran (CBF), cannabiglendol (OH-iso-HHCV), cannabiripsol (CBR), cannbicitran (CBT), or 6-methano-2H-1-benzoxocin-5-mehtnaol.

In other embodiments of the disclosure, the cannabinoid is a cannabidiol or tetrahydrocannabinol isolate. In yet other embodiments of the disclosure, the cannabinoid is a hemp-derived cannabidiol.

In some embodiments, the composition comprises a cannabinoid in an amount of from about 25 mg to about 35 mg.

In some embodiments, the cannabinoid is a mixture of one or more cannabinoid compounds (e.g., a mixture of THC and CBD).

In some embodiments, the composition comprises an excipient that is a co-processed carbohydrate containing mannitol and sorbitol in a 2:1 ratio.

In some embodiments, the composition further comprises a taste enhancing agent and a lubricant. In other embodiments, the composition comprises an excipient that is one or more sugar alcohols.

In some embodiments, the composition dissolves in the oral cavity in about 60 seconds.

In other embodiments, the composition further comprises a terpene.

Another aspect of the disclosure provides a tablet comprising a rapidly dissolving oral composition comprising an effective amount of a cannabinoid and a pharmaceutically acceptable excipient or mixtures of excipients.

In some embodiments, the tablet has a total weight of about 550 mg to about 750 mg, or about 600 mg to about 650 mg.

In some embodiments, the tablet has favorable organoleptic properties or is tasteless.

Yet another aspect of the disclosure provides a method for preparing a rapidly dissolving oral composition comprising: (i) preparing a mixture of powder comprising a cannabinoid; (ii) blending the ingredients prepared in step (i) with at least one tableting excipient; and (iii) compressing the blend of step (ii) into a tablet.

In some embodiments, the tableting excipient comprises a binder, a filler, a taste enhancing agent, a disintegrant, and/or a lubricant. In other embodiments, the tableting excipient is a co-processed carbohydrate containing mannitol and sorbitol in a 2:1 ratio.

In some embodiments, the tablet comprises a cannabinoid that is tetrahydrocannabinol (THC), delta-9-tetrahydrocannabivarin (THCv), 10-Ehtoxy-9-hydroxy-delta-6a-tetrahydrocannabinol, 8,9-dihydroxy-delta-6a-tetrahydrocannbinol, delta-8-tetrahydrocannbinol, delta-8-tetrahydrocannbinolic acid, delta-9-tetrahydrocannbinol-C4 (THC-C4), delta-9-tetrahydrocannbinoic acid A (THCA-A), delta-9-tetrahydrocannbinolic acid B (THCA-B), delta-9-tetrahydrocannbinolic acid-C4 (THCA-C4), delta-9-tetrahydrocannabiorcol (THC-C1), delta-9-tetrahydrocannabiorolic acid (THCA-C1), delta-9-tetrahydrocannabivarinic acid (THCVA), 10-Oxo-delta-6a-tetrahydrocannbinol (OTHC), dehydrocannbifuran (DCBF), delta-9-cis-tetrahydrocannabinol (cis-THC), trhydroxy-delta-9-tetrahydrocannabinol (triOH-THC), 3,4,5,6,-tetrahydro-7-hydroxy-alpha-alpha-2-trimethyl-9-n-propyl-2, cannabidiol (CBDs), including cannabinol (CBN), cannabichromene (CBC), cannabichromenic acid (CBCA), cannabicyclol (CBL), cannabicyclolic acid (CBLA), cannabicyclovarin (CBLV), cannabivarin (CBV), Cannabidivarin (CBDVa), cannabinodiol (CBND), cannabielsion (CBE), cannabicyclol (CBL), cannabicyloic acid (CBLA), cannabitriol (CBT), cannabidiol monomehylerther (CBDM), cannabidiolic acid (CBDA), cannabidiorcol (CBD-C1), cannabielsoic acid B (CBEA-B), cannabielsin (CBE), cannabielsoin acid A (CBEA-A), cannabigerol (CBG), cannabigerol monomethlether (CBGM), cannabigerolic acid (CBGA), cannabigerolic acid (CBGA), cannabigerolic acid monomethlether (CBGAM), cannabigerovarin (CBGV), cannabigerovarinic acid (CBGVA), cannabinodiol (CBND), cannabinodivarin (CBVD), cannabinol methlether (CBNM), cannabinol-C2 (CBN-C2), cannabinol-C4 (CBN-C4), cannabinolic acid (CBNA), cannabiorcool (CBN-C1), cannabivarin (CBV), cannaitriol (CBT), cannabitriolvarin (CBTV), cannabichromanon (CBCF), cannabifuran (CBF), cannabiglendol (OH-iso-HHCV), cannabiripsol (CBR), cannbicitran (CBT), or 6-methano-2H-1-benzoxocin-5-mehtnaol.

In some embodiments, the tablet comprises a cannabinoid in and amount of from about 25 mg to about 35 mg.

### DETAILED DESCRIPTION OF THE DISCLOSURE

For the purposes of promoting an understanding of the principles of the present disclosure, reference will now be made to specific embodiments and specific language will be used to describe the same.

Unless otherwise defined, all terms (including technical and scientific terms) used herein have the same meaning as commonly understood by one having ordinary skill in the art to which this disclosure belongs. It will be further understood that terms, such as those defined in commonly used dictionaries, should be interpreted as having a meaning that is consistent with their meaning in the context of the relevant art and the present disclosure will not be interpreted in an idealized or overly formal sense unless expressly so defined herein.

It will be further understood that a number of aspects and embodiments are disclosed. Each of these has individual benefit and each can also be used in conjunction with one or more, or in some cases all, of the other disclosed aspects and embodiments, whether specifically delineated or not. Accordingly, for the sake of clarity, this description will refrain from repeating every possible combination of the individual aspects and embodiments in an unnecessary fashion. Nevertheless, the specification and claims should be read with the understanding that such combinations are implicitly disclosed, and are entirely within the scope of the disclosure and the claims, unless otherwise specified.

The inventors have discovered new formulations of rapidly dissolving tablets containing active ingredient cannabinoids for targeted fast acting therapeutic use, which can be taken in the oral cavity and can quickly dissolve to increase bioavailability of cannabinoids in the endocannabinoid receptor system. This disclosure further relates to a novel direct compression molecular binding of either tetrahydrocannabinols (THCs), including delta-9-tetrahydrocannabivarin (THCv), 10-Ehtoxy-9-hydroxy-delta-6a-tetrahydrocannabinol, 8,9-dihydroxy-delta-6a-tetrahydrocannabinol, delta-8-tetrahydrocannabinol, delta-8-tetrahydrocannabinolic acid, delta-9-tetrahydrocannabinol-C4 (THC-C4), delta-9-tetrahydrocannabinoic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorolic acid (THCA-C1), delta-9-tetrahydrocannabivarinic acid (THCVA), 10-Oxo-delta-6a-tetrahydrocannbinol (OTHC), dehydrocannbifuran (DCBF), delta-9-cis-tetrahydrocannabinol (cis-THC), trhydroxy-delta-9-tetrahydrocannabinol (triOH-THC), 3,4,5,6,-tetrahydro-7-hydroxy-alpha-alpha-2-trimethyl-9-n-propyl-2, and/or cannabidiols (CBDs), including cannabinol (CBN), cannabichromene (CBC), cannabichromenic acid (CBCA), cannabicyclol (CBL), cannabicyclolic acid (CBLA), cannabicyclovarin (CBLV), cannabivarin (CBV), Cannabidivarin (CBDVa), cannabinodiol (CBND), cannabielsion (CBE), cannabicyclol (CBL), cannabicyloic acid (CBLA), cannabitriol (CBT), cannabidiol monomehylerther (CBDM), cannabidiolic acid (CBDA), cannabidiorcol (CBD-C1), cannabielsoic acid B (CBEA-B), cannabielsin (CBE), cannabielsoin acid A (CBEA-A), cannabigerol (CBG), cannabigerol monomethlether (CBGM), cannabigerolic acid (CBGA), cannabigerolic acid (CBGA), cannabigerolic acid monomethlether (CBGAM), cannabigerovarin (CBGV), cannabigerovarinic acid (CBGVA), cannabinodiol (CBND), cannabinodivarin (CBVD), cannabinol methlether (CBNM), Cannabinol-C2 (CBN-C2), Cannabinol-C4 (CBN-C4), cannabinolic acid (CBNA), cannabiorcool (CBN-C1), cannabivarin (CBV), cannaitriol (CBT), cannabitriolvarin (CBTV), cannabichromanon (CBCF), cannabifuran (CBF), cannabiglendol (OH-iso-HHCV), cannabiripsol (CBR), cannbicitran (CBT), or 6-methano-2H-1-benzoxocin-5-mehtnaol as active ingredients.

### Definitions

Articles “a” and “an” are used herein to refer to one or to more than one (i.e., at least one) of the grammatical object of the article. By way of example, “an element” means at least one element and can include more than one element.

As used herein, the term “and/or” includes any and all combinations of one or more of the associated listed items. It will be further understood that the use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “about” in association with a numerical value means that the numerical value can vary plus or minus by 5% or less of the numerical value.

The human cultivation history of Cannabis dates back 8000 years and the written record of the pharmacologic properties of Cannabis goes back more than 4000 years. Cannabis is a highly variable genus of flowering plant that includes at least three species: Cannabis sativa, Cannabis indica, and Cannabis ruderalis. See U.S. Patent PP27475P2, incorporated herein by reference.

One aspect of the disclosure provides a rapidly dissolving oral composition comprising an effective amount of a cannabinoid and a pharmaceutically acceptable excipient or mixtures of excipients.

As used herein, the term “effective amount” refers to an amount of one or more cannabinoids sufficient for the subject consuming the composition to feel a desired effect (e.g., analgesic effect, anti-inflammatory effect, improvement in mood, or any of the other medicinal effects associated with consuming cannabinoids).

The term “subject” as used herein refers to both human and non-human animals (e.g., domestic and farm animals, non-human primates, and zoo, sports or pet animals, such as dogs, horses, cats, and cows).

As used herein, the term “rapidly dissolving oral composition” refers to a composition (or formulation) that can completely disintegrate in the oral cavity of a subject when the composition comes in contact with saliva. A rapidly dissolving oral composition can be placed in the mouth where it disperses rapidly before swallowing and can disintegrates in 3 minutes or less (e.g., the tablet can disintegrate or dissolve in about 280 seconds, 260 seconds, 240 seconds, 220 seconds, 200 seconds, 180 seconds, 160 seconds, 140 seconds, 120 seconds, 100 seconds, 80 seconds, 60 seconds, 59 seconds, 58 seconds, 57 seconds, 56 seconds, 55 seconds, 54 seconds, 53 seconds, 52 seconds, 51 seconds, 50 seconds, 49 seconds, 48 seconds, 47 seconds, 46 seconds, 45 seconds, 44 seconds, 43 seconds, 42 seconds, 41 seconds, 40 seconds, 39 seconds, 38 seconds, 37 seconds, 36 seconds, 35 seconds, 34 seconds, 33 seconds, 32 seconds, 30 seconds, 25 seconds, or 20 seconds). In some embodiments, the rapidly dissolving oral composition can dissolve in 60 seconds or less.

A rapidly dissolving oral composition can also dissolve by being placed under the tongue (e.g., sublingual).

A rapidly dissolving oral composition can include, but is not limited to, compositions referred to as orodispersibles, rapimelts, quick dissolving compositions, rapid disintegrating compositions, mouth dissolving compositions, oral lyophilisates, melt-in-mouth compositions, mouth dispersing compositions, fast mouth dissolving compositions, fast melting compositions, porous tablets, orodispersing tablets.

In some embodiments, the rapidly dissolving oral composition is a tablet.

A rapidly dissolving oral composition of the disclosure can be evaluated according to following features using methods known in the art: wetting time, hardness, friability test, mechanical strength, uniformity of dispersion, water absorption ratio, taste/mouth sensation, in vitro and in vivo disintegration test, in vitro dissolution test and stability studies and moisture uptake studies. The rapidly dissolving oral composition can also be evaluated for organoleptic features, such as taste-masking properties or having a taste that is sweet. In some embodiments, the rapidly dissolving oral composition is tasteless.

A rapidly dissolving oral composition of the disclosure can contain one or more cannabinoids. For a medical cannabis plant, the key phytochemicals are cannabinoids and terpenes. As used herein, “phytochemical” or “phytocannabinoid” refers to the biologically active chemicals found in cannabis plants. Phytochemicals affect the normal structure or function of the human body and in some cases can treat disease. The mechanisms for the medicinal and psychoactive properties of a cannabis plant, like any medicinal herb, are the pharmacologic effects of its phytochemicals.

The term “cannabinoid” as used herein means any chemical substance that acts upon a cannabinoid receptor. For example, the term “cannabinoid” includes, but is not limited to, cannabinoid ligands such as agonists, partial agonists, inverse agonists, or antagonists, as demonstrated by binding studies and functional assays. A cannabinoid can be identified because its chemical name can include “cannabi” in the name. Where reference is made to a particular cannabinoid herein, each of the acid and/or decarboxylated forms are contemplated as both single molecules and mixtures.

Examples of cannabinoids include, but are not limited to, compounds belonging to any of the following classes of molecules, their derivatives, salts, or analogs: tetrahydrocannabinols (THCs), including delta-9-tetrahydrocannabivarin (THCv), 10-Ehtoxy-9-hydroxy-delta-6a-tetrahydrocannabinol, 8,9-dihydroxy-delta-6a-tetrahydrocannbinol, delta-8-tetrahydrocannabinol, delta-8-tetrahydrocannabinolic acid, delta-9-tetrahydrocannabinol-C4 (THC-C4), delta-9-tetrahydrocannbinoic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorolic acid (THCA-C1), delta-9-tetrahydrocannabivarinic acid (THCVA), 10-Oxo-delta-6a-tetrahydrocannabinol (OTHC), dehydrocannbifuran (DCBF), delta-9-cis-tetrahydrocannabinol (cis-THC), trhydroxy-delta-9-tetrahydrocannabinol (triOH-THC), 3,4,5,6,-tetrahydro-7-hydroxy-alpha-alpha-2-trimethyl-9-n-propyl-2, or cannabidiols (CBDs), cannabinol (CBN), cannabichromene (CBC), cannabichromenic acid (CBCA), cannabicyclol (CBL), cannabicyclolic acid (CBLA), cannabicyclovarin (CBLV), cannabivarin (CBV), Cannabidivarin (CBDVa), cannabinodiol (CBND), cannabielsion (CBE), cannabicyclol (CBL), cannabicyloic acid (CBLA), cannabitriol (CBT), cannabidiol monomehylerther (CBDM), cannabidiolic acid (CBDA), cannabidiorcol (CBD-C1), cannabielsoic acid B (CBEA-B), cannabielsin (CBE), cannabielsoin acid A (CBEA-A), cannabigerol (CBG), cannabigerol monomethlether (CBGM), cannabigerolic acid (CBGA), cannabigerolic acid (CBGA), cannabigerolic acid monomethlether (CBGAM), cannabigerovarin (CBGV), cannabigerovarinic acid (CBGVA), cannabinodiol (CBND), cannabinodivarin (CBVD), cannabinol methlether (CBNM), Cannabinol-C2 (CBN-C2), Cannabinol-C4 (CBN-C4), cannabinolic acid (CBNA), cannabiorcool (CBN-C1), cannabivarin (CBV), cannaitriol (CBT), cannabitriolvarin (CBTV), cannabichromanon (CBCF), cannabifuran (CBF), cannabiglendol (OH-iso-HHCV), cannabiripsol (CBR), cannbicitran (CBT), 6-methano-2H-1-benzoxocin-5-mehtnaol, and isocanabinoids. Examples of non-psychoactive cannabinoids include, but are not limited to, CBD, CBC, CBE, CBG, CBN, CBND, CBT, CBDV, CBGV, and CBCV.

Tetrahydrocannabinol (THC) and tetrahydrocannabivarin (THCV) are the primary psychoactive cannabinoids and are the result of the decarboxylation of tetrahydrocannabinolic acid (THC-A), its acidic precursor. THC-A, (6ar,10ar)-1-hydroxy-6,6,9-trimethyl-3-pentyl-6a,7,8,10a-tetrahydro-6hbenzochromene-2-carboxylic acid, is found in the trichomes of the plant and converted into THC, which actually exists in only minute quantities in the living plant, after harvest and drying. Cannabigerol (CBG), also known as resorcinol, 2-(3,7-dimethyl-2,6-octadienyl)-5-pentyl-, is not considered psychoactive. CBG is known to block the psychoactive effects of THC and is considered medically active in a variety of conditions. Its precursor, cannabigerolic acid (CBG-A) (E)-3-(3,7-Dimethyl-2,6-octadienyl)-2,4-dihydroxy-6pentylbenzoic acid, can have medical effects. Cannabichromene (CBC) and cannabidiol (CBD) are both non-psychoactive and end products of CBG metabolism, like THC, that can be used medically. Cannabichromenic acid (CBC-A), also known as 5-hydroxy-2-methyl-2-(4-methylpent-3-enyl)-7-pentyl-chromene-6carboxylic acid, is acidic cannabichromene.

In some embodiments, the cannabinoid is a cannabinoid isolate. The term “cannabinoid isolate” as used herein refers to cannabinoids that have been isolated or extracted from a Cannabis plant by extraction methods known in the art (e.g., using CO2). Once isolated, the cannabinoid can be refined so that the final crystalized powder is 99% pure of the cannabinoid of interest. For example, a CBD isolate can be a concentrated form of the active cannabidiol in a crystalized powder form. Cannabinoid isolates can have a purity of up to 99% (e.g., 99% or more CBD and no THC or 99% or more THC and no CBD), and can be odorless and tasteless.

In some embodiments, the cannabinoid is a hemp-derived cannabidiol. The term “hemp” as used herein refers to a Cannabis plant that contains less than 0.3% THC.

In some embodiments, the rapidly dissolving oral composition contains about 2 mg to about 150 mg (e.g., 2 mg, 3 mg, 4 mg, 5 mg, 10 mg, 15 mg, 20 mg, 30 mg, 40 mg, 50 mg, 60 mg, 70 mg, 80 mg, 90 mg, 100 mg, 110 mg, 120 mg, 130 mg, 140 mg, or 150 mg) of cannabinoid per composition (e.g., tablet), where the composition can have a total weight of about 550 mg to 750 mg.

In other embodiments, the rapidly dissolving oral composition contains about 25 mg to about 35 mg of cannabinoid per composition (e.g., tablet), where the composition has a total weight of about 550 mg to 625 mg. For example, the rapidly dissolving oral composition can contain about 25.0 mg, 25.1 mg, 25.2 mg, 25.3 mg, 25.4 mg, 25.6 mg, 25.7 mg, 25.8 mg, 25.9 mg, 26.0 mg, 26.1 mg, 26.2 mg, 26.3 mg, 26.4 mg, 26.5 mg, 26.6 mg, 26.7 mg, 26.8 mg, 26.9 mg, 27.0 mg, 27.1 mg, 27.2 mg, 27.3 mg, 27.4 mg, 27.5 mg, 27.6 mg, 27.7 mg, 27.8 mg, 27.9 mg, 28.0 mg, 28.1 mg, 28.2 mg, 28.3 mg, 28.4 mg, 28.5 mg, 28.6 mg, 28.7 mg, 28.8 mg, 28.9 mg, 29.0 mg, 29.1 mg, 29.2 mg, 29.3 mg, 29.4 mg, 29.5 mg, 29.6 mg, 29.7 mg, 29.8 mg, 29.9 mg, 30.0 mg, 30.1 mg, 30.2 mg, 30.3 mg, 30.4 mg, 30.5 mg, 30.6 mg, 30.7 mg, 30.8 mg 30.9 mg, 31.0 mg, 31.1 mg, 31.2 mg, 31.3 mg, 31.4 mg, 31.5 mg, 31.6 mg, 31.7 mg, 31.8 mg, 31.9 mg, 32.0 mg, 32.1 mg, 32.2 mg, 32.3 mg, 32.4 mg, 32.5 mg, 32.6 mg, 32.7 mg, 32.8 mg, 32.9 mg, 33.0 mg, 33.1 mg, 33.2 mg, 33.3 mg, 33.4 mg, 33.5 mg, 33.6 mg, 33.7 mg, 33.8 mg, 33.9 mg, 34.0 mg, 34.1 mg, 34.2 mg, 34.3 mg, 34.5 mg, 34.6 mg, 34.7 mg, 34.8 mg, 34.9 mg, or 35.0 mg. In some embodiments, for example, a rapidly dissolving oral tablet according to the disclosure can have 27.5 mg of cannabinoid (e.g., CBD isolate, THC isolate, CBD/THC isolate mixture, or an isolate of another cannabinoid) in a tablet having a total weight of 600 mg. It will be appreciated that in this example, the rapidly dissolving oral tablet contains about 4.6% (w/w) of cannabinoid.

In other embodiments, the rapidly dissolving oral composition contains a mixture of one or more cannabinoid compounds (e.g., a mixture of CBD and THC).

The term “terpene” as used herein refers to an organic compound that is derived biosynthetically from units of isopentenyl pyrophosphate. Terpene molecules found in plants can be the primary constituents of essential oils and can produce fragrances and smells. Terpenes can be monoterpenoids, sesquiterpenoids, sesterterpenoid, sesquarterpenoids, tetraterpenoids, triterpenoids, tetraterpenoids, Polyterpenoids, isoprenoids, and steroids. Terpenes can be: α-, β-, γ-, oxo-, isomers, or combinations thereof. Examples of terpenes include, but are not limited to, 8-dihydroionone, acetanisole, acetic acid, acetyl cedrene, anethole, anisole, benzaldehyde, bergamotene (α-cis-bergamotene, α-trans-bergamotene), bisabolol (β-bisabolol, α-bisabolol), borneol, bornyl acetate, butanoic/butyric acid, cadinene (α-cadinene, γ-cadinene), cafestol, caffeic acid, camphene, camphor, capsaicin, carene (A-3-carene, delta-3-carene), carotene, carvacrol, carvone, dextro-carvone, laevo-carvone, caryophyllene β-caryophyllene), caryophyllene oxide, castoreum absolute, cedrene (α-cedrene, β-cedrene), cedrene epoxide (α-cedrene epoxide), cedrol, cembrene, chlorogenic acid, cinnamaldehyde (α-amyl-cinnamaldehyde) (α-hexyl-cinnamaldehyde), cinnamic acid, cinnamyl alcohol, citronellal, citronellol, cryptone, curcumene (α-curcumene, γ-curcumene), decanal, dehydrovomifoliol, diallyl disulfide, dihydroactinidiolide, dimethyl disulfide, eicosane/icosane, elemene (8-elemene), estragole, ethyl acetate, ethyl cinnamate, ethyl maltol, eucalyptol/1,8-cineole, eudesmol (α-eudesmol, β-eudesmol, γ-eudesmol), eugenol, euphol, farnesene, farnesol, fenchol (β-fenchol), fenchone, geraniol, geranyl acetate, germacrenes, germacrene B, guaia-1(10),11-diene, guaiacol, guaiene (α-guaiene), gurjunene (α-gurjunene), herniarin, hexanaldehyde, hexanoic acid, humulene (α-humulene, β-humulene), ionol (3-oxo-α-ionol, 13-ionol), ionone (α-ionone, β-ionone), ipsdienol, isoamyl acetate, isoamyl alcohol, isoamyl formate, isoborneol, isomyrcenol, isopulegol, isovaleric acid, isoprene, kahweol, llavandulol, limonene, γ-linolenic acid, linalool, longifolene, α-longipinene, lycopene, menthol, methyl butyrate, 3-mercapto-2-methylpentanal, mercaptan/thiols, β-mercaptoethanol, mercaptoacetic acid, allyl mercaptan, benzyl mercaptan, butyl mercaptan, ethyl mercaptan, methyl mercaptan, furfuryl mercaptan, ethylene mercaptan, propyl mercaptan, thenyl mercaptan, methyl salicylate, methylbutenol, methyl-2-methylvalerate, methyl thiobutyrate, myrcene (p-myrcene, β-myrcene), γ-muurolene, nepetalactone, nerol, nerolidol, trans-nerolido, neryl acetate, nonanaldehyde, nonanoic acid, ocimene, octanel, octanoic acid, p-cymene, pentyl butyrate, phellandrene, phenylacetaldehyde, phenylethanethiol, phenylacetic acid, phytol, pinene (α-pinene, β-pinene), propanethiol, pristimerin, pulegone, quercetin, retinol, rutin, sabinene, sabinene hydrate, cis-sabinene hydrate, trans-sabinene hydrate, safranal, α-selinene, α-sinensal, β-sinensal, β-sitosterol, squalene, taxadiene, terpin hydrate, terpineol, terpine-4-ol, α-terpinene, γ-terpinene, terpinolene, thiophenol, thujone, thymol, α-tocopherol, tonka undecanone, undecanal, valeraldehyde/pentanal, verdoxan, α-ylangene, or umbelliferone.

The rapidly dissolving oral composition can comprise an effective amount of a cannabinoid and a pharmaceutically acceptable excipient or mixtures of excipients.

An excipient is a substance formulated alongside the active ingredient of a composition that can confer a therapeutic enhancement of the active ingredient (e.g., improving disintegration of the composition). Excipients can include a variety of different types of ingredients, including but not limited to, antiadherents, binders, coatings, colors, diluents, disintegrants, fillers, flavors, glidants, lubricants, preservatives, sorbents, and taste enhancing agents. As used herein, non-active ingredients may be referred to generally as an excipient or identified as a type of excipient (e.g., a disintegrant or glidant). It will be appreciated that certain excipients can be categorized as more than one type of excipient (e.g., magnesium sterate is an excipient that can be used as a lubricant or antiadherent).

Acceptable excipients can include, but are not limited to, calcium sulfate, starch, mannitol, kaolin, sorbitol, xylitol, sodium chloride, sodium bicarbonate, citric acid, powdered cellulose derivatives, microcrystalline cellulose, pullulan, silicified microcrystalline cellulose, ammonium bicarbonate, carrageenan, carbohydrates such as Pharmaburst™ (SPI Pharma Inc., New Castle, Del.), magnesium carbonate, tribasic calcium phosphate, calcium sulfate, magnesium oxide, poloxamer, gums, hydroxypropyl methylcellulose, gelatin, and mixtures thereof.

In some embodiments, the excipient is a co-processed carbohydrate. In other embodiments, the excipient is a co-processed carbohydrate containing mannitol and sorbitol in a 2:1 ratio. The co-processed carbohydrate system of the disclosure refers to the system described in U.S. Pat. No. 7,118,765, incorporated herein by reference. In particular, the term “co-processed carbohydrate” as used herein refers to the processing of at least two polyols together to make a single product. For example, mannitol and sorbitol can be co-spray dried by first preparing a single solution of mannitol and sorbitol. The term “co-processed carbohydrate system” shall be construed to include a co-processed carbohydrate plus a disintegrant and a glidant. The term “co-processed carbohydrate system formulation or composition” can include the co-processed carbohydrate system plus one or more cannabinoids to be formed into a tablet.

Disintegrants expand and dissolve when wet (e.g., contacted by saliva) causing an oral dosage form (e.g., a tablet) to break apart and release the active ingredients. Disintegrants can help the oral dosage form to rapidly dissolve. Acceptable disintegrants include, but are not limit to, crospovidone, sodium starch glycolate, sodium carboxymethyl cellulose, calcium carboxymethyl cellulose, croscarmellose sodium, alginic acid, chitosan, methyl cellulose, microcrystalline cellulose, powdered cellulose, lower alkyl substituted hydroxypropyl cellulose, polacrilin potassium, starch, pregelatinized starch, sodium alginate, or combinations thereof.

Glidants are used to promote powder flow by reducing interparticle friction and cohesion. Glidants can be used in combination with lubricants. Acceptable glidants include, but are not limited to, colloidal silica, silica gel, precipitated silica, silicon dioxide, colloidal silicon dioxide, calcium silicate, magnesium silicate, magnesium trisilicate, talc, starch, or combinations thereof.

In some embodiments, the composition further comprises a taste enhancing agent and a lubricant.

Taste enhancing agents, also referred to as sweetners, can make an oral dosage form become more palatable and can mask unpleasant organoleptic properties (e.g., taste and smell). Acceptable taste enhancing agents include, but are not limited to, sucralose, tagatose, aspartame, acesulfame potassium, saccharin, neotame, acesulfame K, and the like, or combinations thereof.

Lubricants can prevent ingredients from clumping together and from sticking to tableting machines. Acceptable lubricants include, but are not limited to, calcium stearate, glyceryl monostearate, glyceryl behenate, glyceryl palmitostearate, hydrogenated vegetable oil, light mineral oil, magnesium stearate, mineral oil, polyethylene glycol, poloxamer, sodium benzoate, sodium lauryl sulfate, sodium stearyl fumarate, stearic acid, talc, zinc stearate, and mixtures thereof.

In some embodiments, the excipient or mixture of excipients comprises one or more sugar alcohols (e.g., mannitol, sorbitol, xylitol, lactitol, or maltitol).

As used herein, the term “direct compression molecular binding” refers to using binder excipients that have the ability to be compressed into a tablet (or other similar oral dosage forms) directly from powdered active and inactive ingredients. Tablet binders are used in formulating a solid oral dosage form and serve to hold the active and inactive ingredients together in a cohesive mix.

Acceptable binders include, but are not limited to, saccharides and their derivatives, sucrose, lactose, starches, cellulose, methyl cellulose, polyvinlypyrrolidone, polyethylene glycol, xylitol, sorbitol, mannitol, or gelatin. Dry binders are added to a powder blend, either following a wet granulation step or as part of a direct powder compression formula.

Another aspect of the disclosure provides a method for preparing a rapidly dissolving oral composition comprising: (i) preparing a mixture of powder comprising a cannabinoid; (ii) blending the ingredients prepared in step (i) with tableting excipients; and (iii) compressing the blend of step (ii) into a tablet.

The term “tableting excipients” as used herein refers to any excipients used to form a tablet. The tableting excipients can include, for example, a filler, a binder, a taste enhancing agent, a disintegrant, and a lubricant. A filler can be any pharmaceutically acceptable filler or diluent, including, but not limited to lactose, starch, dextrose, sucrose, fructose, maltose, mannitol, sorbitol, kaolin, microcrystalline cellulose, powdered cellulose or any combination thereof. The filler can consist of a mixture of water soluble fillers to reduce the chance of unpleasant grittiness when the tablet dissolves in the oral cavity of the patient. The filler can also be a direct compression sugar such as confectioners sugar, dextrates, dextrin, dextrose, fructose, maltose, mannitol, polydextrose, sorbitol, or other sugars and sugar derivatives.

In some embodiments, the tableting excipient is a co-processed carbohydrate containing mannitol and sorbitol in a 2:1 ratio.

It will be appreciated that the tableting method can be used with a variety of different weights of starting materials, and the methods described herein can be scaled up or down to produce the final compositions using methods known in the art.

Another aspect of the present disclosure provides all that is disclosed and illustrated herein.

The following examples are offered by way of illustration and not by way of limitation.

### Example 1: Formulation A

A rapidly dissolving tablet according to the disclosure and referred to herein as Formulation A was produced as follows. A mixture of 600 grams of co-processed carbohydrate system consisting of mannitol and sorbitol in a 2:1 ratio (SPI Pharma Inc., New Castle, Del.) and 300 grams of cannabidiol isolate (Comco LLC, Hanover Mich.) were blended in a LFA Tablet Mixer for 20 minutes. Formulation A (900 grams) the blend was then discharged and tableted using standard tableting procedures.

### Example 2: Formulation B

A rapidly dissolving tablet according to the disclosure and referred to herein as Formulation B was produced as follows. A mixture of 2 kg of co-processed carbohydrate system consisting of mannitol and sorbitol in a 2:1 ratio (SPI Pharma Inc., New Castle, Del.) and 1 kg of cannabidiol (CBD) isolate derived from Hemp (Genncanna, Ky.) were blended in a LFA Tablet Mixer for 20 minutes. Formulation B (3 kg) the blend was then discharged and tableted using standard tableting procedures.

### Example 3: Formulation C

A rapidly dissolving tablet according to the disclosure and referred to herein as Formulation C was produced as follows. A mixture of 2 kg of co-processed carbohydrate system consisting of mannitol and sorbitol in a 2:1 ratio (SPI Pharma Inc., New Castle, Del.), 1 kg of THC derived from R1 (Comco LLC, Hanover, Mich.) were blended in a LFA Tablet Mixer for 20 minutes. Formulation C (3 kg) the blend was then discharged and tableted using standard tableting procedures.

### Example 4: Formulation D

A rapidly dissolving tablet according to the disclosure referred to herein as Formulation D was produced as follows. A mixture of 2 kg of co-processed carbohydrate system consisting of mannitol and sorbitol in a 2:1 ratio (SPI Pharma Inc., New Castle, Del.) and 1 kg of THC and CBD derived from ANEW (Comco LLC, Hanover, Mich.) were blended in a LFA Tablet Mixer for 20 minutes. Formulation D (3 kg) the blend was then discharged and tableted using standard tableting procedures.

### Example 5: Tablet Compositions for Formulations A-D

The rapidly dissolving tablets referred to herein as Formulations A-D can have a final tablet composition as described in Table 1.

TABLE 1
Composition of a Tablet of Formulations A-D

| Ingredient | mg/tablet | % (w/w) |
|---|---|---|
| SPI Pharma Pharmaburst | 500 | 82.6-83.3 |
| Mannitol/Sorbitol | 67.5 | 11.2-11.3 |
| Cannabinoid Isolate | 27.5-32.5 | 4.6-5.4 |
| Sweetener (sucralose) | 3 | 0.5 |
| Lubricant (sodium Stearyl | 2 | 0.3 |
| fumarate) | | |
| Total Tablet Weight | 600-605 | 100 |

Compaction profiles, that is, determination of maximum tablet hardness values, friability values, and disintegration time for each of the Formulations A, B, C and D can be determined as described in U.S. Pat. No. 7,118,765, incorporated herein by reference. 100 percent increase in tablet hardness can be attained using the co-spray dried carbohydrate system. All formulations can achieve a high tablet hardness at 11.5 KP and with no observed capping. The co-spray dried carbohydrate system is superior as compared to simply dry blended ingredients, in preparing tablets with at least a 100 percent increase in tablet hardness over dry blended ingredients.

Friability of the Formulations. Afriability value of about 1 percent or less is desirable for tablets in order for them to withstand the stress of handling during production, packaging and transport. The formulations described herein can achieve low friability levels. Friability can remain low and even decrease as tablet hardness increased. Co-spray dried carbohydrate system can achieve a much lower friability percent as compared with the same ingredients prepared as dry blends.

Disintegration Times in Oral Cavity for Different formulations. Disintegration time can range from about 30 seconds to about 60 seconds varying with tablet hardness up to about 8 KP. Over-the-counter quick dissolve tablets provide a reference point having a tablet hardness of about 1.5 KP and a disintegration time of about 42 seconds.

Any patents or publications mentioned in this specification are indicative of the levels of those skilled in the art to which the disclosure pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present disclosure is well adapted to carry out the objects and obtain the ends and advantages mentioned, as well as those inherent therein. The present disclosure described herein is presently representative of preferred embodiments, are exemplary, and are not intended as limitations on the scope of the disclosure. Changes therein and other uses will occur to those skilled in the art which are encompassed within the spirit of the disclosure as defined by the scope of the claims.

1. A rapidly dissolving oral composition comprising an effective amount of a cannabinoid and a pharmaceutically acceptable excipient or mixtures of excipients.

2. The composition of claim 1, wherein the cannabinoid is tetrahydrocannabinol (THC), including delta-9-tetrahydrocannabivarin (THCv), 10-Ehtoxy-9-hydroxy-delta-6a-tetrahydrocannabinol, 8,9-dihydroxy-delta-6a-tetrahydrocannbinol, delta-8-tetrahydrocannabinol, delta-8-tetrahydrocannabinolic acid, delta-9-tetrahydrocannabinol-C4 (THC-C4), delta-9-tetrahydrocannabinoic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorolic acid (THCA-C1), delta-9-tetrahydrocannabivarinic acid (THCVA), 10-Oxo-delta-6a-tetrahydrocannbinol (OTHC), dehydrocannbifuran (DCBF), delta-9-cis-tetrahydrocannabinol (cis-THC), trhydroxy-delta-9-tetrahydrocannabinol (triOH-THC), 3,4,5,6,-tetrahydro-7-hydroxy-alpha-alpha-2-trimethyl-9-n-propyl-2, cannabidiol (CBD), cannabinol (CBN), cannabichromene (CBC), cannabichromenic acid (CBCA), cannabicyclol (CBL), cannabicyclolic acid (CBLA), cannabicyclovarin (CBLV), cannabivarin (CBV), Cannabidivarin (CBDVa), cannabinodiol (CBND), cannabielsion (CBE), cannabicyclol (CBL), cannabicyloic acid (CBLA), cannabitriol (CBT), cannabidiol monomehylerther (CBDM), cannabidiolic acid (CBDA), cannabidiorcol (CBD-C1), cannabielsoic acid B (CBEA-B), cannabielsin (CBE), cannabielsoin acid A (CBEA-A), cannabigerol (CBG), cannabigerol monomethlether (CBGM), cannabigerolic acid (CBGA), cannabigerolic acid (CBGA), cannabigerolic acid monomethlether (CBGAM), cannabigerovarin (CBGV), cannabigerovarinic acid (CBGVA), cannabinodiol (CBND), cannabinodivarin (CBVD), cannabinol methlether (CBNM), cannabinol-C2 (CBN-C2), cannabinol-C4 (CBN-C4), cannabinolic acid (CBNA), cannabiorcool (CBN-C1), cannabivarin (CBV), cannaitriol (CBT), cannabitriolvarin (CBTV), cannabichromanon (CBCF), cannabifuran (CBF), cannabiglendol (OH-iso-HHCV), cannabiripsol (CBR), cannbicitran (CBT), or 6-methano-2H-1-benzoxocin-5-mehtnaol.

3. The composition of claim 1, wherein the cannabinoid is a cannabidiol or tetrahydrocannabinol isolate.

4. The composition of claim 1, wherein the cannabinoid is a hemp-derived cannabidiol.

5. The composition of claim 1, wherein the cannabinoid is in an amount of from about 25 mg to about 35 mg.

6. The composition of claim 1, wherein the cannabinoid is a mixture of one or more cannabinoids.

7. The composition of claim 6, wherein the cannabinoid is a mixture comprising THC and CBD.

8. The composition of claim 1, wherein the excipient is a co-processed carbohydrate containing mannitol and sorbitol in a 2:1 ratio.

9. The composition of claim 1, wherein the composition further comprises a taste enhancing agent and a lubricant.

10. The composition of claim 1, wherein the excipient comprises one or more sugar alcohols.

11. The composition of claim 1, wherein the composition dissolves in the oral cavity in about 60 seconds.

12. The composition of claim 1, wherein the composition further comprises a terpene.

13. A tablet comprising the composition of claim 1.

14. The tablet of claim 13, wherein the total weight of the tablet is about 600 to about 650 mgs.

15. The tablet of claim 13, wherein the tablet is tasteless.

16. A method for preparing a rapidly dissolving oral composition comprising: (i) preparing a mixture of powder comprising a cannabinoid; (ii) blending the ingredients prepared in step (i) with at least one tableting excipient; and (iii) compressing the blend of step (ii) into a tablet.

17. The method of claim 16, wherein the tableting excipient comprise a binder, a filler, a taste enhancing agent, a disintegrant, and/or a lubricant.

18. The method of claim 16, wherein the tableting excipient is a co-processed carbohydrate containing mannitol and sorbitol in a 2:1 ratio.

19. The method of claim 16, wherein the cannabinoid is tetrahydrocannabinol (THC), delta-9-tetrahydrocannabivarin (THCv), 10-Ehtoxy-9-hydroxy-delta-6a-tetrahydrocannabinol, 8,9-dihydroxy-delta-6a-tetrahydrocannbinol, delta-8-tetrahydrocannbinol, delta-8-tetrahydrocannabinolic acid, delta-9-tetrahydrocannabinol-C4 (THC-C4), delta-9-tetrahydrocannabinoic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorolic acid (THCA-C1), delta-9-tetrahydrocannabivarinic acid (THCVA), 10-Oxo-delta-6a-tetrahydrocannabinol (OTHC), dehydrocannabifuran (DCBF), delta-9-cis-tetrahydrocannabinol (cis-THC), trhydroxy-delta-9-tetrahydrocannabinol (triOH-THC), 3,4,5,6,-tetrahydro-7-hydroxy-alpha-alpha-2-trimethyl-9-n-propyl-2, cannabidiol (CBD), cannabinol (CBN), cannabichromene (CBC), cannabichromenic acid (CBCA), cannabicyclol (CBL), cannabicyclolic acid (CBLA), cannabicyclovarin (CBLV), cannabivarin (CBV), cannabidivarin (CBDVa), cannabinodiol (CBND), cannabielsion (CBE), cannabicyclol (CBL), cannabicyloic acid (CBLA), cannabitriol (CBT), cannabidiol monomehylerther (CBDM), cannabidiolic acid (CBDA), cannabidiorcol (CBD-C1), cannabielsoic acid B (CBEA-B), cannabielsin (CBE), cannabielsoin acid A (CBEA-A), cannabigerol (CBG), cannabigerol monomethlether (CBGM), cannabigerolic acid (CBGA), cannabigerolic acid (CBGA), cannabigerolic acid monomethlether (CBGAM), cannabigerovarin (CBGV), cannabigerovarinic acid (CBGVA), cannabinodiol (CBND), cannabinodivarin (CBVD), cannabinol methlether (CBNM), cannabinol-C2 (CBN-C2), cannabinol-C4 (CBN-C4), cannabinolic acid (CBNA), cannabiorcool (CBN-C1), cannabivarin (CBV), cannaitriol (CBT), cannabitriolvarin (CBTV), cannabichromanon (CBCF), cannabifuran (CBF), cannabiglendol (OH-iso-HHCV), cannabiripsol (CBR), cannbicitran (CBT), or 6-methano-2H-1-benzoxocin-5-mehtnaol.

20. The method of claim 16, wherein the tablet comprises a cannabinoid in an amount of about 25 mg to about 35 mg per tablet.
